(19) United States
(12) Patent Application Publication
(10) Pub. No.: US 2011/0028542 A1
(43) Pub. Date: Feb. 3, 2011

(54) COMPOSITIONS FOR PREVENTION OR TREATMENT OF ANOREXIA-CACHEXIA SYNDROME AND USES THEREOF

(76) Inventors: Jean Legault, Chicoutimi (CA); Andre Pichette, Chicoutimi (CA)

Correspondence Address: GOUDREAU GAGE DUBUC, 2000 MCGILL COLLEGE, SUITE 2200, MONTREAL, QC, H3A 3H3, CA

(21) Appl. No.: 12/602,406

(22) PCT Filed: May 6, 2008
(86) PCT No.: PCT/CA08/00865

§ 371 Date: Sep. 28, 2010

Related U.S. Application Data
(60) Provisional application No. 60/941,117, filed on May 31, 2007.

(51) Int. Cl. A61K 31/355; A61K 31/015; A61K 31/085; A61K 31/05; A61K 31/353; A61P 35/00; A61P 21/00; A61P 19/00; A61P 3/00

(52) U.S. Cl. 514/458; 514/766; 514/718; 514/731; 514/456

(57) ABSTRACT

A pharmaceutical composition for 1) increasing weight gain; and/or 2) decreasing weight loss; and/or 3) increasing appetite in a subject in need thereof, comprising purified β-caryophyllene together with a pharmaceutically acceptable carrier. Also provided are pharmaceutical compositions for use in the prevention or treatment of Anorexia-Cachexia Syndrome, the pharmaceutical composition comprising purified β-caryophyllene together with a pharmaceutically acceptable carrier.

### CROSS REFERENCE TO RELATED APPLICATIONS

This application claims priority under 35 U.S.C. §119(e), of U.S. provisional application Ser. No. 60/941,117, filed on May 31, 2007. The above document is incorporated herein in its entirety by reference.

### FIELD OF THE INVENTION

The present invention relates to β-caryophyllene. More specifically, the present invention is concerned with compositions comprising β-caryophyllene and uses thereof in the prevention, treatment or delay of Anorexia-Cachexia Syndrome.

### BACKGROUND OF THE INVENTION

Anorexia-Cachexia Syndrome (ACS), which includes cancer cachexia is a complex disorder characterized by an involuntary progressive loss of weight, that may be associated with at least one additional symptom such as muscle wasting, anorexia, asthenia (lack of energy and strength), anemia and alterations in immune function. It is a significant cause of morbidity and mortality, occurring in up to 80% of patients with advanced cancer (Table 1), and responsible for death in up to 20% of cases. Different tumours display varying propensities to induce cachexia. ACS, is most commonly seen in subjects with gastrointestinal, lung and prostate cancers, in contrast to hematological and breast malignancies where it is rare. Weight loss is associated with both reduced quality of life, and shortened life expectancy, with death occurring when subjects have lost around 30% of their premorbid weight. Additionally, ACS patients have a lower chance of responding to an underlying treatment (e.g., chemotherapy) and are more prone to toxic side-effects (Stewart et al., 2006).

TABLE 1
THE COMMONEST MALIGNANCIES IN WHICH
CACHEXIA DEVELOPS AS PART OF THE CLINICAL COURSE

| | Patients |
|---|---|
| Malignancy | with cachexia |
| Gastric cancer | 85% |
| Pancreatic cancer | 83% |
| Non-small cell lung cancer | 61% |
| Small cell lung cancer | 57% |
| Prostate cancer | 56% |
| Colon cancer | 54% |
| Non-Hodgkin's lymphoma (unfavorable) | 48% |
| Sarcoma | 40% |
| Acute non-lymphocytic leukemia | 39% |
| Breast cancer | 36% |

Stewart, G. D., Skipworth, R. JE, Fearon, K. CH. (2006). Cancer cachexia and fatigue. Clinical Medicine, 6: 140-143.

Although it is most common in cancer, ACS may appear in nearly all forms of life-threatening illnesses including AIDS and other viral infections as well as Congestive Heart Failure. Most frequent symptoms associated with ACS include anorexia, muscle wasting, loss of lean body mass, anemia, weakness, chronic fatigue, asthenia, and multiple organ dysfunction. Anorexia in ACS is different from anorexia nervosa which is a mental illness. It is defined by a loss or decrease in appetite which translates into inability to eat and its consequent weight loss. Cachexia is defined as a state of general ill health and malnutrition marked by weakness and emaciation. In contrast to anorexia, the weight loss in cachexia involves not only loss of fat and muscle but also of bone mass. Progressive loss of body weight during cachexia results from an important reduction in the adipose tissues and from skeletal muscles (Inui et al., 2002).

Indeed, several changes at the level of fat tissues and skeletal muscles were observed during cancer cachexia. Firstly, glycerol and fatty acid turnover is increased compared to healthy subjects or cancer patients without loss of weight. Evidences also show that lipolysis is increased. On the other hand, several studies indicate that protein synthesis of the scrawny muscles is decreased while their degradation is increased. Furthermore, numerous works demonstrate that activity and expression of enzymes involved in the degradation of proteins (see ubiquitin-proteosome pathway) were significantly increased. Consistently, the increase in catabolism of the fat tissues and scrawny muscles suggests that specific factors produced by the tumor and the hosts are involved in cancer cachexia (Tisdale, on 2005, Inui et al., 2002).

Many drugs and nutritional therapies have been proposed and used to treat, at least partly, ACS. Pharmacological agents proven useful to treat cancer-associated ACS include progestational agents (e.g., megestrol acetate and medroxyprogesterone) and glucocorticoids (e.g., dexamethasone, and prednisolone). These agents can stimulate appetite and potentially ameliorate weight loss but are either generally short lasting (in the case of glucocorticoids) and/or show undesirable side effects (e.g., thrombotic episodes and oedema in the case of progestational agents and myopathy, diabetes mellitus, cataract formation, gastric ulceration osteoporosis etc., in the case of glucocorticoids). Anabolic steroids, cannabinoids, anti-serotoninergic agents, cyproheptadine, metoclopramide, eicosapentaenoic acid (EPA) and pentoxifylline have been evaluated in cancer ACS patients providing mixed results and limited usefulness.

Thus, in view of the fact that ACS remains difficult to treat and greatly impact on patients quality of life, response to treatment as well as life expectancy, there remains a need for alternative compositions and uses thereof in the prevention or treatment of such condition. The present invention addresses these and other needs.

The present description refers to a number of documents, the content of which is herein incorporated by reference in their entirety.

### SUMMARY OF THE INVENTION

Accordingly, the present invention provides methods, pharmaceutical compositions, formulations and kits that can be used for treating or preventing ACS in mammals. More particularly, it was surprisingly discovered that compositions comprising β-caryophyllene reduce weight loss associated with ACS, including cancer ACS and increase food consumption. In addition, beneficial effects of the compositions of the present invention are not limited to the reduction of weight loss in ill subjects. Indeed, it was found that compositions comprising β-caryophyllene, when administered to healthy subjects can significantly increase weight gain as well as appetite.

Thus, in a first aspect, the present invention provides a method for preventing or treating ACS in a subject in need thereof comprising administering a therapeutically effective amount of a composition comprising β-caryophyllene.

In a second aspect, the present invention provides a use of a composition comprising β-caryophyllene for the preparation of a medicament for preventing or treating, ACS.

In a further related aspect, the present invention provides a composition for preventing or treating ACS comprising β-caryophyllene together with a suitable pharmaceutical carrier.

In another aspect, the present invention provides a package or kit for preventing or treating ACS comprising a composition comprising β-caryophyllene together with instructions for preventing or treating ACS in a subject in need thereof.

In an embodiment, the above compositions, methods, uses or packages of the present invention are for preventing or treating cancer ACS. In another embodiment, the above compositions, methods uses or packages are for preventing or treating AIDS ACS.

In a specific embodiment, the composition of the present invention is administered or used prior to the onset of ACS, as a preventive measure. In another embodiment, the composition of the present invention is administered or used once the subject has been diagnosed with ACS. In a particular embodiment, the composition of the present invention is administered or used once the subject has lost more than 5% of his/her weight prior to the onset of the disease. In an embodiment the composition of the present invention is administered or used once the subject has lost more than 5% of his/her weight within a 6-month period. In yet another embodiment, the composition of the present invention is administered or used once the subject has lost at least 10% of his/her weight prior to the onset of the disease. In a related embodiment the composition of the present invention is administered or used once the subject has lost at least 10% of his/her weight within a 6-month period.

In a further specific embodiment, the composition of the present invention is used in combination with another drug which treats an underlying disease or condition (e.g., an antitumoral agent or anti-viral agent). In another embodiment, the compositions of the present invention are co-administered with the drug treating the underlying disease or condition (e.g., an antitumoral agent or an anti-viral agent).

In another embodiment, the composition of the present invention is administered or used in combination with one or more other drugs or food supplements, or both, which are normally used for the prevention or treatment of ACS. In an embodiment the one or more drugs and/or food supplements are selected from the following classes: glucocorticoids (e.g., dexamethasone, methylprednisolone) progestational agents (e.g., megestrol acetate, medroxyprogesterone), cannabinoids (e.g., dronabinol), anabolic steroids (e.g., Fluoxymestrone, Oxandrolone), antiserotoninergic agents (e.g., cyproheptadine, ondansetron, mirtazapine), prokinetic agents (e.g., metoclopramide), n-3 fatty acids and others (NSAIDs (e.g., ibuprofen), eicosapentaenoic acid, pentoxifylline, melatonin, thalidomide, etc).

In another embodiment, the composition of the present invention is formulated, administered or used in combination with an antitumoral agent selected from: carboplatin, melphalan, cyclophosphamide, lomustine, chlorambucil, carmustine and cisplatine; etoposide, mitoxantrone, daunorubicin and doxorubicin; 5-fluorouracile, floxuridine, gemcitabine, mercaptopurine, tioguanine, fludarabine, cytarabine, pemetrexed, raltitrexed and methotrexate; paclitaxel and docetaxel; vinblastine, vincristine and vindesine, vinorelbine; selected from the group consisting of aclarubicin, and mitomycin C; tamoxiphen and tyrphostin; steroids and glucocordicoid hormones.

In a specific embodiment, the composition of the present invention is used and/or co-administered in combination with an antitumoral agent selected from paclitaxel and docetaxel.

In an embodiment, administration or use of the composition of the present invention is continued once the treatment for the underlying disease or condition has been suspended or terminated. In a specific embodiment, the composition of the present invention is first used and/or co-administered with an anti-tumoral agent intravenously and then at a subsequent point in time, when the antitumoral treatment is suspended or terminated, the composition of the present invention is administered orally. In yet another specific embodiment, the composition administered orally is in the form of a syrup. Oral compositions of the present invention can advantageously easily be self-administered by the subject in a non clinical environment such as at the subject's home.

In another aspect, the present invention provides a method of 1) increasing weight gain; and/or 2) decreasing weight loss; and/or 3) increasing appetite comprising administering an effective amount of composition comprising β-caryophyllene.

In a related aspect, the present invention provides a composition for 1) increasing weight gain; and/or 2) decreasing weight loss; and/or 3) increasing appetite in a subject in need thereof comprising β-caryophyllene together with a suitable carrier.

The present invention also provides a use of a composition comprising β-caryophyllene for the preparation of a medicament for 1) increasing weight gain; 2) decreasing weight loss and/or 3) increasing appetite.

In another embodiment, the present invention provides a food, drink or food supplement comprising purified β-caryophyllene in an amount not naturally found in such food or drink for 1) increasing weight gain; 2) decreasing weight loss and/or 3) increasing appetite in a subject in need thereof.

Thus, the present invention also provides a use of purified β-caryophyllene as a food supplement for 1) increasing weight gain; 2) decreasing weight loss and/or 3) increasing appetite.

In another aspect, the present invention provides a package or kit for 1) increasing weight gain; and/or 2) decreasing weight loss; and/or 3) increasing appetite comprising a composition comprising β-caryophyllene together with instructions for 1) increasing weight gain; and/or 2) decreasing weight loss; and/or 3) increasing appetite in a subject in need thereof.

In an embodiment, the compositions, formulations and kits of the present invention comprising β-caryophyllene comprise purified β-caryophyllene. Accordingly, in an embodiment methods and uses as provided herein comprise purified β-caryophyllene.

In an embodiment, the compositions formulations and kits of the present invention comprising β-caryophyllene comprise substantially pure β-caryophyllene. Similarly, in an embodiment methods and uses as provided herein comprise substantially pure β-caryophyllene. In yet a further embodiment, the compositions, formulations, kits, methods derived there from and uses thereof comprise synthetic β-caryophyllene.

In U.S. provisional application No. 60/941,117, the Applicants have provided stable formulations for water-insoluble sesquiterpenes such as β-caryophyllene which have a generally weak hydrosolubility. In addition, it was surprisingly discovered that β-caryophyllene is sensitive to acidity and that in certain solubilizers found to be appropriate for liquid formulations, β-caryophyllene is oxidized into β-caryophyllene oxide which is an irritant.

Thus, the present invention also provides a composition comprising in addition to purified β-caryophyllene one or more antioxidants and/or one or more solubilizers selected from the group consisting of oils (e.g., olive oil), PEG 400, a derivative of castor oil and ethylene oxide, and polysorbate 80.

In another specific embodiment, the present invention provides a composition comprising in addition to purified β-caryophyllene one or more antioxidants selected from the group consisting of: vitamin E, a hydrophilic vitamin E analog, alpha tocopherol acetate, butylated hydroxytoluene (BHT) and butylated hydroxyanisole (BHA). In other specific embodiments of the composition, the antioxidant is 6-Hydroxy-2,5,7,8-tetramethylchroman-2-carboxylic acid. In other specific embodiments of the composition, the antioxidant is vitamin E. In other specific embodiments of the composition, the solubilizer is a polysorbate. In other specific embodiments of the composition, the polysorbate is polysorbate 80. In other specific embodiments of the composition, the solubilizer is a derivative of castor oil and ethylene oxide. In other specific embodiments of the composition, the solubilizer is an oil. In other specific embodiments of the composition, the oil is olive oil.

In other specific embodiments, the pharmaceutical composition further comprises an isotonic agent selected from the group consisting of dibasic sodium phosphate, sodium bicarbonate, calcium chloride, potassium chloride, sodium lactate, glycerol, sorbitol, xylitol, sodium chloride, dextrose, a Ringer's solution, a lactated Ringer's solution and a mixture of dextrose and a mixture thereof. In other specific embodiments, the composition comprises from about 0.01 mg/mL to about 100 mg/mL of purified beta-caryophyllene, from about 0.0001% to about 5% v/v of antioxidant, from about 0.01% to about 20% v/v of solubilizer, and an isotonic agent. In other specific embodiments, the composition comprises about 1% v/v of purified beta-caryophyllene, about 0.1% v/v of antioxidant, about 5% v/v of solubilizer, and about 93.5% v/v of an isotonic agent.

In other specific embodiments of the composition is in a daily dosage comprising from about 0.001 mg/kg to about 5000 mg/kg. In other specific embodiments of the composition is in a daily dosage comprising from about 1 mg/kg to about 5000 mg/kg. In other specific embodiments of the composition is in a daily dosage comprising from about 1 mg/kg to about 1000 mg/kg. In other specific embodiments of the composition is in a daily dosage comprising from about 1 mg/kg to about 300 mg/kg. In other specific embodiments of the composition is in a daily dosage comprising from about 0.001 mg/kg to about 300 mg/kg. In other specific embodiments of the composition is in a daily dosage comprising from about 1 mg/kg to about 80 mg/kg.

In other specific embodiments of the composition, the antioxidant is vitamin E and the solubilizer is polysorbate 80. In other specific embodiments of the composition, the antioxidant is vitamin E and the solubilizer is olive oil. In other specific embodiments of the composition, the antioxidant is 6-Hydroxy-2,5,7,8-tetramethylchroman-2-carboxylic acid and the solubilizer is polysorbate 80. In other specific embodiments of the composition, the antioxidant is a combination of 6-Hydroxy-2,5,7,8-tetramethylchroman-2-carboxylic acid and of vitamin E. In other specific embodiments of the composition, the isotonic agent is sodium chloride.

In other specific embodiments of the composition, the antioxidant is 6-Hydroxy-2,5,7,8-tetramethylchroman-2-carboxylic acid, the solubilizer is polysorbate 80, and the isotonic agent is sodium chloride. In other specific embodiments of the composition, the antioxidant is vitamin E, the solubilizer is polysorbate 80, and the isotonic agent is sodium chloride.

In another specific embodiment, the composition of the present invention comprises in addition to β-caryophyllene a combination of a water soluble antioxidant and a water insoluble antioxidant. In an embodiment, the water soluble antioxidant is selected from Trolox®, ascorbic acid, hypophosphoric acid, potassium metabisulfite and sodium sulfite. In an embodiment, the water insoluble antioxidant is selected from tocopherols, carotenoids and Vitamin E. In a specific embodiment the water insoluble antioxidant is Vitamin E.

In other specific embodiments of the composition, the composition is in a capsule. In other specific embodiments of the composition, the composition is in a liquid form. In other specific embodiments of the composition, the composition is in a soft gel capsule. In other specific embodiments of the composition, the composition has an enteric coating.

Other objects, advantages and features of the present invention will become more apparent upon reading of the following non-restrictive description of illustrative embodiments thereof, given by way of example only with reference to the accompanying drawings.

### BRIEF DESCRIPTION OF THE DRAWINGS

In the appended drawings:

FIG. 1 shows the structure of β-caryophyllene ((1R, 4E, 9S)-4-11,11-trimethyl-8-methylenebicyclo[7.2.0]undec-4-ene, CAS regustry number [87-44-5]). Numbering is in accordance with Collado (1989).

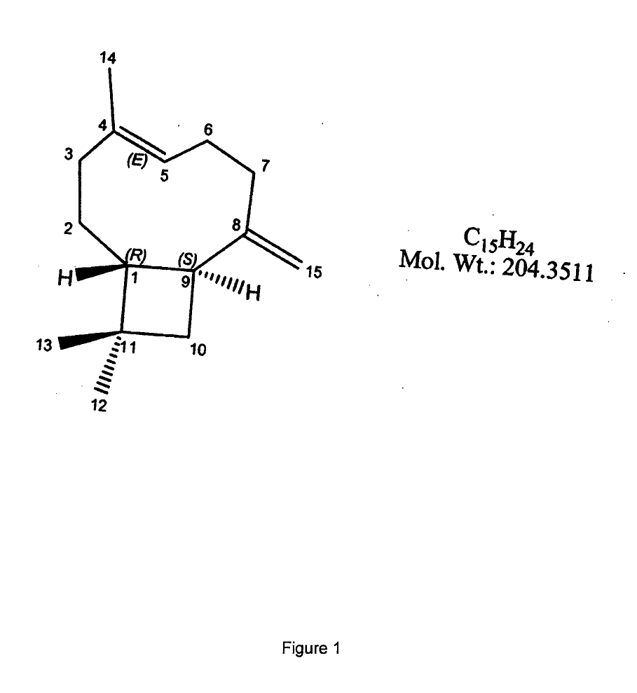

FIG. 2 shows that Taxotere® treatment induces loss of weight on day 7. Tumor LLC-bearing mice were treated on day 1 to 4 with saline or with 5, 10 or 15 mg/kg of Taxotere®. The weight of mice was determined every day and the percentage of loss or gain was calculated with regards to initial weight on day 0. *The asterisk* indicates a significant difference compared with control saline; the results were analyzed by the Kruskal-Wallis One Way Analysis of variance on Ranks and Dunn's Method. P values of 0.05 or less were considered as statistically significant;

FIG. 3 shows that β-caryophyllene (identified as FPL-99) protects tumor LLC-bearing mice against ACS induced by Taxotere® on day 7. Tumor LLC-bearing mice were treated on day 1 to 4 with saline or 15 mg/kg Taxotere® alone or in combination with 6.25, 12.5 or 25 mg/kg of β-caryophyllene. The mice were also treated with 25 mg/kg of β-caryophyllene alone. The weight of mice was determined every day and the percentage of loss or gain of weight was calculated with regards to initial weight on day 0. The asterisk* indicates a significant difference compared with control saline; the results were analyzed by the Kruskal-WallisOne Way Analysis of Variance on Ranks and Dunn's method. ‡ significantly different from Taxotere® 15 mg/kg (p≦0.001); One Way Analysis of Variance on Ranks and Duncan's Method;

FIG. 4 shows that β-caryophyllene (identified as FPL-99) protects tumor LLC-bearing mice against ACS induced by Taxotere® on day 7. The mice were considered cachectic when the loss of weight was superior to 20% with regards to initial weight (day 0). Tumor LLC-bearing mice were treated on day 1 to 4 with saline or 15 mg/kg of Taxotere® alone or in combination with, 12.5 or 25 mg/kg of β-caryophyllene. The weight of mice was determined every day and the percentage of loss or gain of weight was calculated;

FIG. 5 shows that β-caryophyllene (identified as FPL-99) decreases anorexia induced by Taxotere® on tumor LLC-bearing mice. The mice were treated with saline, β-caryophyllene (25 mg/kg), Taxotere® (15 mg/kg) or Taxotere® (15 mg/kg) combined with β-caryophyllene (25 mg/kg) on day 1 to 4. The consumption of food was measured every day. The results are expressed as the percentage of change of food consumption on day 5 to 7;

FIG. 6 shows that β-caryophyllene (identified as FPL-99) protects against anorexia induced by Taxotere® treatment on tumor LLC-bearing mice. The mice were treated with 1) saline, 2) Taxotere® alone (15 mg/kg) 3) β-caryophyllene alone (6.25 mg/Kg; 12.5 mg/Kg and 25 mg/Kg) or 4) a combination of β-caryophyllene (6.25 uM; 12.5 uM and 25 mg/Kg) and Taxotere® (15 mg/kg) on day 1 to 4. The consumption of food was measured every day up to Day 14. The results are expressed as the percentage of change of food consumption on day 1 to 14;

FIG. 7 shows that β-caryophyllene (identified as FPL-99) induces weight gain on day 7. Tumor LLC-bearing mice were treated with saline or 6.25, 12.5 or 25 mg/kg of β-caryophyllene on day 1 to 4. The weight of mice was determined every day and the percentage of gain of weight was calculated with regards to initial weight on day 0.

FIG. 8 shows that β-caryophyllene (identified as FPL-99) increases the body weight of healthy mice. Healthy mice were treated with saline (control) or 12.5 and 25 mg/kg of β-caryophyllene on day 1 to 4. The weight of mice was determined every day and the percentage of gain of weight was calculated with regards to initial weight on day 0. The asterisk* indicates a significant difference compared with control saline; the results were analyzed by the Kruskal-Wallis One Way Analysis of Variance and Dunnett's Method. P values of 0.05 or less were considered as statistically significant;

FIG. 9 shows that β-caryophyllene (identified as FPL-99) increases the body weight of healthy mice. Healthy mice were treated with saline (control) or 6.25, 12.5 and 25 mg/kg of β-caryophyllene on day 1 to 4. The weight of mice was determined every day and the percentage of gain of weight was calculated with regards to initial weight on day 0;

FIG. 10 shows the effect of β-caryophyllene on the loss of body weight induced by turpentine. The results are expressed as percentage of loss of weight with regards to initial weight on day 1. The mice were treated orally with olive oil (vehicle) or β-caryophyllene (50 or 300 mg/kg) on day 1 to 3. Each value is the mean of at least eight different measures. The vertical bars represent the standard deviation of each data point. Different letters (a-c) denote a significant difference (RM one way ANOVA analysis and post hoc Student-Newman-Keuls Method; p<0.05); and

FIG. 11 shows the effect of some monoterpenes and sesquiterpenes on the loss of body weight induced by turpentine. Three monoterpenes (beta-myrcene, limonene, and beta-pinene) and four sesquiterpenes (alpha-humulene, isocaryophyllene, trans-nerolidol, and beta-bisabolol) were tested. The results are expressed as percentage of loss of weight with regards to initial weight on day 1. The mice were treated orally with olive oil (vehicle) or terpenes (300 mg/kg) on day 1 to 3. Different letters (a-c) differ significantly (RM one way ANOVA analysis and post hoc Student-Newman-Keuls Method; p<0.05.

### DESCRIPTION OF ILLUSTRATIVE EMBODIMENTS

Described herein are studies using various mouse model systems for anorexia-cachexia syndrome including cancer anorexia-cachexia syndrome (CACS) which show that β-caryophyllene is able to significantly decrease weight loss and/or increase weight gain and/or increase appetite. In addition, it was discovered that β-caryophyllene is able to promote weight gain in healthy subject thereby indicating that the weight gain promoting activity of β-caryophyllene is not limited to ACS in ill subjects but has a more general effect on growth and/or weight. This activity is very specific to β-caryophyllene as other sesquiterpenes, including isomers of β-caryophyllene (namely alpha-humulene and isocaryophyllene) are unable to significantly protect against the loss of weight in a mouse model of ACS.

Accordingly, the methods, compositions, formulations and uses of the present invention are particularly useful in the prevention or treatment of ACS symptoms including but not limited to weight loss. In addition, the methods, compositions, formulations and uses of the present invention are also particularly useful for generally promoting weight gain, reducing weight loss and/or increasing appetite in ill but also in healthy subjects.

Cachexia is a syndrome characterized by an involuntary loss of weight and may include one or more of progressive loss of both fat and skeletal muscle, refractoriness of weight loss to increase nutritional input, elevated resting energy expenditure (REE), decreased protein synthesis, altered carbohydrate metabolism, hyper-catabolism/increased degradation of muscle via the ATP-ubiquitin-proteasome pathway of proteolysis and of adipose tissue via lipolysis, asthenia, anemia, chronic fatigue, nausea, and loss of bone mass (Muscaritoli M., et al., European J Cancer 42:31-41, 2006). Typically, at least 5% or 5 pounds of pre-illness body weight must have been lost before the patient is diagnosed with cachexia.

Of course one or more of the above symptoms may or may not be present in a given subject depending on the underlying disease or condition associated with it and of the treatment already received by the subject for treating the underlying disease or condition. The above symptoms or physiological conditions may also be present at various degrees. Cachexia may or may not be associated with anorexia.

Anorexia is a medical term for appetite loss. Manifestations of anorexia include a decreased sense of taste and smell of food, early satiety, a decreased sense of hunger and even outright aversion of food.

The terminology Anorexia-Cachexia Syndrome is a generic term used by physician as a diagnostic of patients having either anorexia or cachexia. As used herein therefore, ACS designates anorexia or cachexia. Diseases or conditions associated with or likely to be associated with ACS, include but are not limited to, cancer, immunodeficiency disorders such as AIDS, other infectious diseases including viral, bacterial and parasitic diseases, sepsis, rheumatoid arthritis and chronic diseases of the bowel, liver, kidneys, lungs and heart including congestive heart failure and chronic organ failure. It can also manifest itself as a condition in aging or as a result of physical traumas and burn injuries.

An “underlying disease or condition” is a disease or condition that is associated with ACS or that is likely to be associated with ACS.

As used herein Cancer Anorexia-Cachexia-Syndrome (CACS) is intended to include any form of cancer associated with ACS or likely to be associated with ACS. Non-limiting examples of cancers that are most often associated with ACS include gastric cancer, pancreatic cancer, non-small cell lung cancer, small cell lung cancer lung cancer, prostate cancer, colon cancer, non-Hodgkin's lymphoma, sarcoma, acute non-lymphocytic leukemia and breast cancer.

The methods, compositions formulations and uses described herein are suitable for both humans and animals, preferably mammals.

Thus, as used herein, the term “subject” in the context of the present invention relates to any mammal including a mouse, rat, pig, monkey and horse. In a specific embodiment, it refers to a human. A “subject in need thereof” or a “patient” in the context of the present invention is intended to include any subject that will benefit or that is likely to benefit from the compositions and pharmaceutical compositions of the present invention. In an embodiment, a subject in need thereof is a subject diagnosed with ACS or having a disease or condition that is likely to be associated with ACS. Subjects having cancer or AIDS are examples of likely candidates. The likelihood of developing ACS can be determined for instance with the prevalence of cancer as presented in Table 1. In an embodiment, a subject in need thereof is a subject suffering from cancer. In another embodiment, the subject in need thereof is a subject suffering from cancer but which has not yet developed ACS. In an embodiment, a subject in need thereof is a subject suffering from an immunodeficiency such as AIDS. In a further embodiment, the subject in need thereof is a subject which has lost at least 5%, 8%, 10%, 12%, 15% or more of his/her initial weight prior to the onset of ACS. In another embodiment, the subject in need thereof is a subject which has lost at least 5%, 8%, 10%, 12%, 15% or more of his/her weight within a six-month period. In a further embodiment, the subject in need thereof is a subject that is desirous or increasing his/her appetite and/or weight. In yet another embodiment, a subject in need thereof is a subject undergoing therapy for the underlying disease or condition which is associated with ACS or likely to be associated with ACS.

Thus, in one aspect of the present invention the pharmaceutical composition comprising β-caryophyllene is administered prior to the onset of ACS as a preventive measure. In another aspect of the present invention the pharmaceutical composition of the present invention is administered in combination with a drug or drugs used to treat the underlying disease or condition. In a further aspect, the composition of the present invention is administered once the subject has been diagnosed with ACS. In another embodiment, the composition of the present invention is administered in combination with one or more other drugs or food supplements used for the prevention and/or treatment of ACS.

Accordingly, the present invention further provides a method of preventing and/or treating ACS comprising administering a therapeutically effective amount of β-caryophyllene in combination with a further drug useful to prevent and/or treat ACS. Non limiting examples of drugs that treat, prevent or delay ACS include glucocorticoids (e.g., dexamethasone, methylprednisolone) progestational agents (e.g., megestrol acetate, medroxyprogesterone), cannabinoids (e.g., dronabinol), anabolic steroids (e.g., Fluoxymestrone, Oxandrolone), antiserotoninergic agents (e.g., cyproheptadine, ondansetron, mirtazapine), prokinetic agents (e.g., metoclopramide) and others (NSAIDs (ibuprofen), eicosapentaenoic acid, pentoxifylline, melatonin, thalidomide, ibuprofen, natural oils and fatty acids, etc).

The present invention further provides a method of preventing and/or treating ACS comprising administering a therapeutically effective amount of β-caryophyllene together with an antitumoral agent from a class selected from the group consisting of alkylating agent, antimetabolite, antimitotic, antibiotic, immunotherapy and hormone. Non limiting examples of antitumoral agents that can be administered prior to, concomitantly with or after the pharmaceutical compositions of the present invention comprising β-caryophyllene include carboplatin, melphalan, cyclophosphamide, lomustine, chlorambucil, carmustine and cisplatine (alkylating agents); etoposide, mitoxantrone, daunorubicin and doxorubicin (topoisomerase II inhibitors); 5-fluorouracile, floxuridine, gemcitabine, mercaptopurine, tioguanine, fludarabine, cytarabine, pemetrexed, raltitrexed and methotrexate (antimetabolites); paclitaxel and docetaxel (antimitotic); vinblastine, vincristine and vindesine, vinorelbine (vinca alkaloids); selected from the group consisting of aclarubicin, and mitomycin C (antibiotics); tamoxiphen and tyrphostin (kinase inhibitors); steroid and glucocordicoid hormones.

In prior U.S. Patent Application Nos. 60/941,117 and U.S. Ser. No. 10/488,682, (which are herein incorporated by reference in their entirety) the present Applicants have shown that compositions comprising β-caryophyllene are useful in potentiating the activity of certain antitumoral agents, thereby improving cancer treatment. This potentiating activity of β-caryophyllene is independent from its weight gain promoting activity, as well as its anti-cachectic and anti-anorexic activities. Thus, under certain conditions, it could be highly beneficial for the subject to be treated with an antitumoral agent who's activity is potentiated by β-caryophyllene. Thus, the patient treated with the right antitumoral agent would benefit from the anti-cachectic activity and anti-anorexic activity of β-caryophyllene but from a more efficient cancer treatment.

Examples of such antitumoral agents which are potentiated by β-caryophyllene include antitumoral agents from the class of antimitotic, vinca alkaloids and alkylating agents. Non-limiting examples of antimitotic agents which are potentiated by β-caryophyllene include docetaxel and paclitaxel. Non-limiting examples of alkylating and vinca alkaloids agents which are potentiated by β-caryophyllene include cisplatine, and vinorelbine.

As indicated above, the composition of the present invention may be used in combination with other drugs or food supplement used to: 1) prevent or treat an underlying disease or condition; and/or 2) prevent or treat ACS. The combination of prophylactic/therapeutic agents and/or compositions of the present invention may be administered or co-administered (e.g., consecutively, simultaneously, at different times) in any conventional dosage form. Co-administration in the context of the present invention refers to the administration of more than one therapeutic in the course of a coordinated treatment to achieve an improved clinical outcome. Such co-administration may also be coextensive, that is, occurring during overlapping periods of time. For example, a first agent may be administered to a patient before, concomitantly, before and after, or after a second active agent is administered. The agents may in an embodiment be combined/formulated in a single composition and thus administered at the same time. In an embodiment, the one or more active agent(s) of the present invention is used/administered in combination with one or more agent(s) currently used to prevent or treat the underlying disease or condition.

The pharmaceutical compositions of the present invention preferably comprise purified β-caryophyllene. As used herein, the term “purified” refers to a molecule (i.e., β-caryophyllene) having been separated from one or more components of the composition in which it was originally contained (e.g., natural extracts or chemical synthesis contaminants). A “purified β-caryophyllene” molecule is a molecule that is lacking in most other components (e.g., 70, 75, 80, 85, 90, 95, 96, 97, 98, 99, 100% free of contaminants). “Substantially pure β-caryophyllene” is intended to include β-caryophyllene molecules that are at least 95% free of contaminants. The terms “purified β-caryophyllene” or “substantially pure β-caryophyllene” are intended to include both β-caryophyllene purified from natural extracts and chemically synthesized β-caryophyllene. By opposition, the term “crude” or “semi-purified” means molecules that have not been separated from other components of the composition from which β-caryophyllene originates (e.g., semi purified natural extracts, essential oils etc.). For the sake of brevity, the units (e.g. 66, 67 . . . 81, 82, 91, 92% . . . ) have not been specifically recited but are considered nevertheless within the scope of the present invention. Of course, a person skilled in the art would appreciate that in the context of pharmaceutical compositions it is preferable, although not essential, that β-caryophyllene be as pure as possible (i.e., substantially free of contaminants). Purity can be measured using any appropriate method such as by column chromatography, HPLC, etc.

The terms “treat/treating/treatment” and “prevent/preventing/prevention” as used herein, refers to eliciting the desired biological response, i.e., a therapeutic and prophylactic effect, respectively. Hence “treat/treating/treatment” in the expression “treating ACS” is meant to refer to any partial or complete reduction in involuntary weight loss and optionally of one or more additional symptoms of a pre-existing ACS in a subject. Similarly the terminology “prevent” in the expression “prevent ACS” is meant to refer to any delay in the onset of involuntary weight loss and optionally of one or more additional symptoms of ACS or a reduced, delayed or slowed progression or severity of involuntary weight loss and optionally of one or more additional symptoms of ACS or any partial or complete avoidance of involuntary weight loss and optionally of one or more additional symptoms of ACS in a subject.

In accordance with the subject invention, the therapeutic or prophylactic effect comprises one or more of 1) an increase in weight gain; 2) a decrease in weight loss; 3) an increase in food consumption; and 4) an increase in the subject's appetite. In addition, the therapeutic or prophylactic effect may comprise an amelioration of one or more other symptoms associated with ACS (progressive loss of both fat and skeletal muscle, refractoriness of weight loss to increased nutritional input, elevated resting energy expenditure (REE), decreased protein synthesis, altered carbohydrate metabolism, hyper-catabolism/increased degradation of muscle via the ATP-ubiquitin-proteasome pathway of proteolysis and of adipose tissue via lipolysis, asthenia, anemia, chronic fatigue, nausea, loss of bone mass and an increased survival time of the affected subject, following administration of a pharmaceutical composition comprising β-caryophyllene of the present invention.

The articles “a,” “an” and “the” are used herein to refer to one or to more than one (i.e., to at least one) of the grammatical object of the article.

The terms “including” and “comprising” are used herein to mean, and are used interchangeably with, the phrases “including but not limited to” and “comprising but not limited to”.

The terms “such as” is used herein to mean, and is used interchangeably herein with “such as but not limited to”.

### Routes of Administration, Dosages and Formulations/Carriers

Route of Administration

Pharmaceutical compositions can also be administered by routes such as orally, nasally, rectally, topically, intravenously, intramuscularly, subcutaneously, sublingually, intrathecally, intraperitoneally, intra-articularly or intradermally.

The route of administration can depend on a variety of factors, such as the environment and therapeutic goals. Thus, the compositions of the present invention can be formulated in any desired way e.g., in or as a feed, a food, a liquid, an elixir, an aerosol, a spray, a tablet, a capsule, a gel, a nanosuspension, a nanoparticle a microgel, a cream an ointment or a suppository.

Hence in specific embodiments, when the composition of the present invention is for oral administration, the composition is in a capsule such as a soft gel capsule. In other specific embodiments when the composition of the present invention is for oral administration, the composition has an enteric coating. In other specific embodiments when the composition of the present invention is for oral administration, the composition is an oil-based syrup. In other specific embodiments of the pharmaceutical composition when the composition of the present invention is for oral administration, the composition comprises pure β-caryophyllene without any solubilizer.

Dosage

The compositions and formulations of the present invention are administered in amounts and at a frequency sufficient to prevent, treat and/or ameliorate ACS. A subject's progress can be determined by measuring and observing changes in the appearance of the subject (e.g., visible and measurable changes in body mass); body composition (e.g., lean body mass), and/or by determining relevant clinical markers which are well-known in the art (e.g., ubiquitine-proteasome pathway makers, cytokines expression, etc). The determination, measurement, and evaluation of such characteristics and markers associated with clinical progress are known to those of ordinary skill in the art.

Any amount of a pharmaceutical composition can be administered to a subject. The dosages will depend on many factors including the mode of administration and the age of the subject. Typically, the amount of β-caryophyllene contained within a single dose will be an amount that effectively prevents or treats involuntary weight loss and optionally, one or more additional symptoms associated with ACS without inducing significant toxicity i.e., muscle wasting, progressive loss of both fat and skeletal muscle, refractoriness of weight loss to increased nutritional input, elevated resting energy expenditure (REE), decreased protein synthesis, altered carbohydrate metabolism, hyper-catabolism/increased degradation of muscle via the ATP-ubiquitin-proteasome pathway of proteolysis and of adipose tissue via lipolysis, asthenia, anemia, chronic fatigue nausea, and loss of bone mass.

As used herein the term “therapeutically effective amount” is meant to refer to an amount effective to achieve the desired therapeutic effect while avoiding adverse side effects. Typically and depending on the route of administration, in accordance with the present invention, β-caryophyllene can be administered to subjects in doses ranging from 0.001 to 5000 mg of β-caryophyllene per kg of body weight each day.

The dosage will also be adapted by the clinician in accordance with conventional factors such as the extent of the disease and different parameters from the patient and may depend on whether the subject is also taking other drugs for treating an underlying disease or condition (e.g., cancer or AIDS).

The therapeutically effective amount of the pharmaceutical composition of the present invention may also be measured directly. The effective amount may be given daily or weekly or fractions thereof. Typically, a pharmaceutical composition of the invention can be administered in an amount providing about 0.001 up to about 5000 mg of β-caryophyllene per kg of body weight each day (e.g., 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 10, 15, 20, 25, 30, 50, 100, 200; 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000 mg/kg/day).

For example, the maximum recommended starting dose (MRSD) for human is calculated by establishing the No Observed Adverse Effect Level (NOAEL, see Guidance for Industry and Reviewers. December 2002). Many concentrations of the formulation described above have been tested on mice, namely 6.25 mg/kg, 12.5 mg/kg, 25 mg/kg, 50 mg/kg, 300 mg/kg, 1000 mg/kg and 5000 mg/kg. No undesirable effects have been observed with any of these doses. The NOAEL is thus 5000 mg/kg for mice.

The dose may be scaled up to a human equivalent dose (HED) for starting clinical trials using published conversion tables which provide a conversion factor from mice to human of 12.3. For example, A NOAEL of 5000 mg/kg for that species is equivalent to 24.39 mg/kg in human.

Dosages may be provided in either a single or multiple dosage regimen. For example, in some embodiments the effective amount is a dose that ranges from about 1000 to about 5000 mg/kg/day, about 1000 to about 3000 mg/kg/day, 300 to about 1000 mg/kg/day, about 0.01 to about 10 mg/kg/day, about 0.01 to about 5 mg/kg/day, from about 0.02 to about 1 mg/kg/, about 0.02 to about 2 mg/kg/day, about 0.02 to about 3 mg/kg/day, about 0.02 to about 4 mg/kg/day, about 0.14 to about 35 mg/kg/week, about 0.14 to about 42 mg/kg/week, about 0.14 to about 49 mg of β-caryophyllene every other day. In specific embodiments, β-caryophyllene used to prevent, treat or delay ACS is administered in a dosage of about 0.5 to about 2 mg/kg to a human. An average human adult would thus receive about 30 to about 120 mg and thus about 3 to 12 mL of a β-caryophyllene formulation at a concentration of 10 mg/mL. An average human adult would thus receive about 60 to about 240 mg and thus about 6 to 24 mL of a beta-caryophyllene formulation at a concentration of 10 mg/mL.

These are simply guidelines since the actual dose must be carefully selected and titrated by the attending physician based upon clinical factors unique to each patient. The optimal daily dose will be determined by methods known in the art and will be influenced by factors such as the age of the patient as indicated above and other clinically relevant factors. In addition, patients may be taking medications for other diseases or conditions.

### Carriers/Vehicles

The pharmaceutical composition may further comprise a pharmaceutically acceptable carrier or excipient. As used herein “pharmaceutically acceptable carrier” or “excipient” includes any and all solvents, buffers, dispersion media, coatings, antibacterial and antifungal agents, isotonic and absorption delaying agents, and the like that are physiologically compatible. The carrier is selected for administration by the selected route of administration. The use of such media and agents for pharmaceutically active substances is well known in the art (Rowe et al., Handbook of pharmaceutical excipients, 2003, 4^{th} edition, Pharmaceutical Press, London UK). Except insofar as any conventional media or agent is incompatible with the active compound, use thereof in the pharmaceutical compositions of the invention is contemplated.

Non-limiting pharmaceutically suitable materials that may be incorporated in pharmaceutical preparations of the present invention include solubilizing/diluting agents, antioxidants, enteric coatings, absorption enhancers, pH adjusting agents and buffers, dispersing agents, coatings, antibacterial and antifungal agents, absorption delaying agents, osmolarity adjusters, isotonic agents, preservative agents, stabilizers, surfactants, thickening agents, solvents, co-solvents, emollients, coloring agents, wetting agents and ligands/pilote/targeting molecules. Methods for preparing appropriate formulations are well known in the art (see e.g., Hendrickson, 2005).

Solubilizing agents useful for the present invention encompass polyoxyethylene-sorbitan-fatty acid esters, polyoxyethylene fatty acid esters, PEG glyceryl fatty acid esters, propylene glycol mono- or di-fatty acid esters, sorbitan fatty acid esters, polyoxyethylene-polyoxypropylene co-polymers, glycerol triacetate, monoglycerides, acetylated monoglycerides, polysorbate glycerol fatty acid esters, acetylated esters of fatty acids, acacia, carbomer copolymer, carbomer Interpolymer, cholesterol, diethanolamine aluminium monostearate, carboxy methyl cellulose, sodium desoxycholate, egg yolk phospholipid, hydrolyzed gelatin, lecithin, lanolin alcohols, poloxamer, povidone, sodium dodecyl sulphate, sorbitol, oils such as vegetable oils or animal oils (see relevant sections of USP-NF and Nema, 1997). Non-limiting examples of vegetable oils include canola, corn, flax seeds, cotton seeds, soybean, walnut, pine nut, peanut, grape seed, sunflower, safflower, olive, coconut, palm oil etc). Non-limiting examples of animal oils include fish, seal oil and castor oil. In more specific embodiments, it includes any polysorbate including polysorbate 20, 21, 40, 60, 61, 65, 80, 81 and 85; Brij™ (polyoxyethyleneglycol alkyl ether having a polar side of 10 to 100 monomers) and Cremophor™ (e.g. Cremophor™ EL (derivative of castor oil and ethylene oxide); Cremophor™ A6 (Polyethylene glycol 260 mono(hexadecyl/octadecyl)ether and 1-Octadecanol) and Cremophor™ A25 (Polyethylene glycol 1100 mono(hexadecyl/octadecyl)ether).

The solubilizers containing polyoxyethylene chains such as polysorbates, PEG, and Brij™ are susceptible to formation of peroxydes by radicalar reactions catalyzed by light and oxygen. In specific embodiments, solubilizers used in beta-caryophyllene formulations are PEG400, Cremophor™ EL, polysorbate 60 and polysorbate 80.

Antioxidants useful for formulations of the present invention include plant extracts (i.e. fruit, vegetable and/or leguminous extracts), algae extracts, microorganisms extracts such as yeast extracts and its derivatives, ferments, proteolysis hydrolysates, peptides, animal derivative extracts and synthetic compounds. More particularly, such ingredients include Ethylbisiminomethylguaiacol manganese chloride; dipalmitoyl hydroxyproline, dimethylmethoxy chromanol; bioflavonoid hesperidin olive leaf extract, ubiquinone, super-oxide dismutase, flavanols, isoflavones, furfuryladenine, panthenol, lipoic acid, niacinamide, melatonin, catalase, glutathione, polyphenols, cysteine, allantoin, kinetin, squalane, grape seed extract and camellia sinensis extract, ascorbic acid and its derivatives (ascorbyl palmitate, magnesium ascorbyl phosphate, sodium ascorbyl phosphate) vitamin E and its derivatives (e.g. α-tocopherol, δ-tocopherol, γ-tocopherol, tocopheryl acetate, a hydrophilic vitamin E analog such as 6-Hydroxy-2,5,7,8-tetramethylchroman-2-carboxylic acid (Trolox™), alpha-tocopherol acetate, alpha-tocopheryl polyethylene glycol succinate, alpha-tocopherol palmitate), butylated hydroxytoluene (BHT), butylated hydroxyanisole (BHA), hypophosphorous acid, monothioglycerol, potassium metabisulfite, propyl gallate, sodium bisulfite, sodium formaldehyde sulfoxylate, sodium metabisulfite, sodium sulfite, sodium thiosulfate and sulfur dioxide (see USP-NF and Nema, 1997).

The terms “preservative agent” as used herein are meant to refer to any ingredient capable of retarding or preventing microbial or chemical spoilage and protecting against discoloration. Without being so limited, they include benzalkonium chloride, benzethonium chloride, benzyl alcohol, butylparaben, chlorobutanol, chlorocresol, cresol, ethylparaben, methylparaben, myristyl gamma-picolinium chloride, phenol, phenoxyethanol, phenylmercuric Acetate, phenylmercuric nitrate, propylparaben, thimerosal (see Nema, 1997).

The terms “isotonic agent” as used herein are meant to refer to ingredients capable of adjusting osmolarity. Without being so limited, they include dibasic sodium phosphate, sodium bicarbonate, calcium chloride, potassium chloride, sodium lactate, glycerol, sorbitol, xylitol, sodium chloride, dextrose, a Ringer's solution, a lactated Ringer's solution and a mixture of dextrose and a mixture thereof (see relevant sections of USP-NF). A lactated Ringer's solution is a solution of recently boiled distilled water containing 39 mmol/L of sodium ion, 42 mmol/L of chloride ion, 0.6 mmol/L of bicarbonate ion, 1.4 mmol/L of potassium ion and 42 mmol/L of calcium ion—the same concentrations as their occurrence in body fluids. Ingredients are: NaCl 2.25 g, KCl 0.105 g, CaCl₂ 0.12 g, NaHCO₃ 0.05 g.

The term “solvent” as used herein is meant to refer to ingredients capable of facilitating the solubilization of an active within the formulation. Without being so limited, it includes water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. Intravenous vehicles may include fluid and nutrient replenishers, electrolyte replenishers, such as those based upon Ringer's dextrose, and the like.

Parenteral Formulations

In cases where parenteral administration is elected as the route of administration, pharmaceutical compositions of the present invention may be provided to patients in combination with additional pharmaceutically acceptable sterile aqueous or non-aqueous solvents, suspensions or emulsions. Formulations to be used for in vivo administration are preferably sterile. This is readily accomplished, for example, by filtration through sterile filtration membranes.

Pharmaceutically acceptable carriers for parenteral formulations include sterile aqueous solutions or dispersions and sterile powders for the extemporaneous preparation of sterile injectable solutions or dispersion. Aqueous solvents include water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. Intravenous vehicles may include fluid and nutrient replenishers, electrolyte replenishers, such as those based upon Ringer's dextrose, and the like.

Formulating water insoluble molecules such as β-caryophyllene into a pharmaceutical formulation often presents important challenges. In particular, β-caryophyllene has weak hydrosolubility, as its molecular structure is devoid of an hydrophilic moiety.

A frequently used method of formulating a weakly hydrosoluble molecule in an aqueous carrier involves the use of ethanol, an organic solvent that is appropriate for intravenous injectable formulation when it is used at small concentrations. This approach however disadvantageously results in a rapid phase separation when water is added to comply with the FDA requirement that ethanol be contained at a maximum concentration of 80%.

In addition, Applicants have recently shown that β-caryophyllene is sensitive to acidity and that in certain solubilizers found to be appropriate for liquid formulations, β-caryophyllene oxidizes into β-caryophyllene oxide. β-caryophyllene oxide is considered to be an irritant and is thus considered herein to be an impurity. The concentration of such an impurity in the formulations of the present invention is low. In an embodiment, the pharmaceutical composition of the present invention comprises purified β-caryophyllene, an antioxidant, and a solubilizer selected from the group consisting of a PEG400, a derivative of castor oil and ethylene oxide, and polysorbate 80.

In other specific embodiments of the pharmaceutical composition, the antioxidant is selected from the group consisting of vitamin E, a hydrophilic vitamin E analog, alpha tocopherol acetate, butylated hydroxytoluene (BHT) and butylated hydroxyanisole (BHA). In other specific embodiments of the pharmaceutical composition, the antioxidant is 6-Hydroxy-2,5,7,8-tetramethylchroman-2-carboxylic acid. In other specific embodiments of the pharmaceutical composition, the antioxidant is vitamin E. In other specific embodiments of the pharmaceutical composition, the solubilizer is a polysorbate. In other specific embodiments of the pharmaceutical composition, the polysorbate is polysorbate 80. In other specific embodiments of the pharmaceutical composition, the solubilizer is a derivative of castor oil and ethylene oxide (e.g., Cremophor EL®).

In certain embodiments, the present invention encompasses the use of an inert or noble gas for filling the headspace of a container enclosing a formulation of the present invention. Any inert or noble gas can be used for this purpose such as Argon, helium, neon, krypton, xenon and radon.

In other specific embodiments, the pharmaceutical composition comprises from about 0.01 mg/mL to about 100 mg/mL of β-caryophyllene, from about 0.0001% to about 5% v/v of antioxidant, from about 0.01% to about 20% v/v of solubilizer, and an isotonic agent. In other specific embodiments, the pharmaceutical composition comprises about 1% v/v of β-caryophyllene, about 0.1% v/v of antioxidant, about 5% v/v of solubilizer, and about 93.5% v/v of an isotonic agent.

In other specific embodiments of the pharmaceutical composition, the antioxidant is vitamin E and the solubilizer is polysorbate 80. In other specific embodiments of the pharmaceutical composition, the antioxidant is 6-Hydroxy-2,5,7,8-tetramethylchroman-2-carboxylic acid and the solubilizer is polysorbate 80. In other specific embodiments of the pharmaceutical composition, the antioxidant is a combination of 6-Hydroxy-2,5,7,8-tetramethylchroman-2-carboxylic acid and of vitamin E. In other specific embodiments of the pharmaceutical composition, the isotonic agent is sodium chloride. In other specific embodiments of the pharmaceutical composition, the antioxidant is 6-Hydroxy-2,5,7,8-tetramethylchroman-2-carboxylic acid, the solubilizer is polysorbate 80, and the isotonic agent is sodium chloride. In other specific embodiments of the pharmaceutical composition, the antioxidant is vitamin E, the solubilizer is polysorbate 80, and the isotonic agent is sodium chloride.

Without being so limited, it is assumed that a perfusion can administer about 200 ml/hour for up to about 5 hours to an average adult of about 60 kg. Without being so limited, it is assumed that injections can administer as much as 1000 ml within 20 minutes. In accordance with a specific embodiment, formulations of the present invention contain about 10 mg/ml of β-caryophyllene. 1000 ml of such formulation injected to an average adult weighting 60 kg contain 10 000 mg of beta-caryophyllene namely 167 mg/kg.

Oral Formulations

Formulations suitable for oral administration can consist of (a) liquid solutions, such as an effective amount of active agent(s)/composition(s) suspended in diluents/solubilizers, such as water, vegetable or animal oils, saline or PEG 400; (b) capsules such as soft shell capsules, sachets or tablets, each containing a predetermined amount of the active ingredient, as liquids, solids, granules or gelatin; (c) suspensions in an appropriate liquid; and (d) suitable emulsions.

Aqueous solutions suitable for oral use are prepared by dissolving the active compound(s)/composition(s) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents. Examples of non-aqueous solvents are alcohol, benzyl benzoate, butyl alcohol, polyethylene glycol, propylene glycol, N,N-dimethylacetamide, ethyl oleate, oleyl oleate, glyceryl trioleate, glyceryl dioleate, glyceryl monooleate, cetyl alcohol, stearyl alcohol, capric acid, undecenoic acid, undecanoic acid, lauric acid, oleic acid, synthetic glycerides of saturated fatty acids with 8 to 12 carbon atoms, polyoxyethylene derivatives of glycerol, bees' wax, glycerin, mineral oil, vegetable oil such as but not limited to corn oil, cottonseed oil, peanut oil, canola oil, sesame oil, safflower oil, soybean oilarachis oil, castor oil, linseed oil, soya bean oil, sunflower seed oil, olive oil, fish liver oil, and any combination thereof (see Nema, 1997).

Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

In an embodiment, pharmaceutical compositions of the present invention may be provided to subjects in an encapsulated form such as a soft shell encapsulation. For instance, an aqueous formulation comprising beta-caryophyllene can be encapsulated in such a soft shell.

Enteric coatings can further be used on capsules of the present invention to resist prolonged contact with the strongly acidic gastric fluid, but dissolve in the mildly acidic or neutral intestinal environment. Without being so limited, cellulose acetate phthalate, Eudragit™ and hydroxypropyl methylcellulose phthalate (HPMCP) can be used in enteric coatings of pharmaceutical compositions of the present invention. Cellulose acetate phthalate concentrations generally used are 0.5-9.0% of the core weight. The addition of plasticizers improves the water resistance of this coating material, and formulations using such plasticizers are more effective than when cellulose acetate phthalate is used alone. Cellulose acetate phthalate is compatible with many plasticizers, including acetylated monoglyceride; butyl phthalybutyl glycolate; dibutyl tartrate; diethyl phthalate; dimethyl phthalate; ethyl phthalylethyl glycolate; glycerin; propylene glycol; triacetin; triacetin citrate; and tripropionin. It is also used in combination with other coating agents such as ethyl cellulose, in drug controlled-release/time-release preparations.

The present invention is illustrated in further details by the following non-limiting examples.

### Example 1

### β-Caryophyllene Inhibits Cachexia Induced by Lewis Lung Tumor Combined with Taxotere®

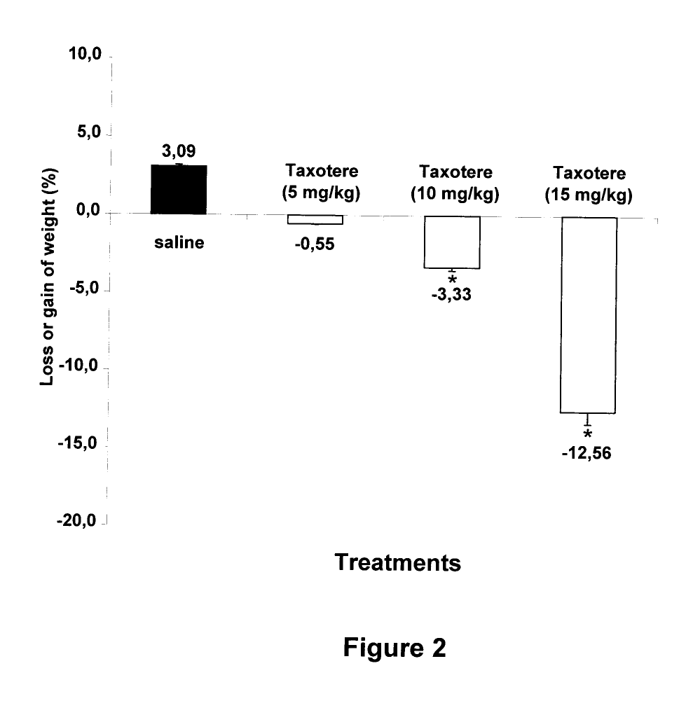

β-caryophyllene, a sesquiterpene, was shown to protect mice against cachexia induced by Lewis lung carcinoma and Taxotere® (docetaxel). Tumor-bearing mice were treated intravenously with 5, 10 and 15 mg/kg of Taxotere® on day 1 to 4 alone or in combination with 12.5 or 25 mg/kg of β-caryophyllene. Ten mice were included in each group and saline, administered intravenously, was used as control. The weight of mice was determined every day. In FIG. 2, the results are expressed as loss or gain of weight with regards to initial weight. On day 7, the results show that treatment

TABLE 2
β-caryophyllene protects tumor LLC-bearing mice against cachexia
induced by Taxotere ® on day 7

| | Loss of | |
|---|---|---|
| | weight >20% | Toxicity* |
| Treatments | (n = 10) | (%) |
| Saline | 0 | 0 |
| Taxotere ® 15 mg/kg | 7 on 10 | 70 |
| β-caryophyllene 6.25 mg/kg | 0 | 0 |
| β-caryophyllene 12.5 mg/kg | 0 | 0 |
| β-caryophyllene 25 mg/kg | 0 | 0 |
| β-caryophyllene 6.25 mg/kg + | 3 on 10 | 30 |
| Taxotere ® 15 mg/kg | | |
| β-caryophyllene 12.5 mg/kg + | 3 on 10 | 30 |
| Taxotere ® e 15 mg/kg | | |
| β-caryophyllene 25 mg/kg + Taxotere ® | 1 on 10 | 10 |
| 15 mg/kg | | |

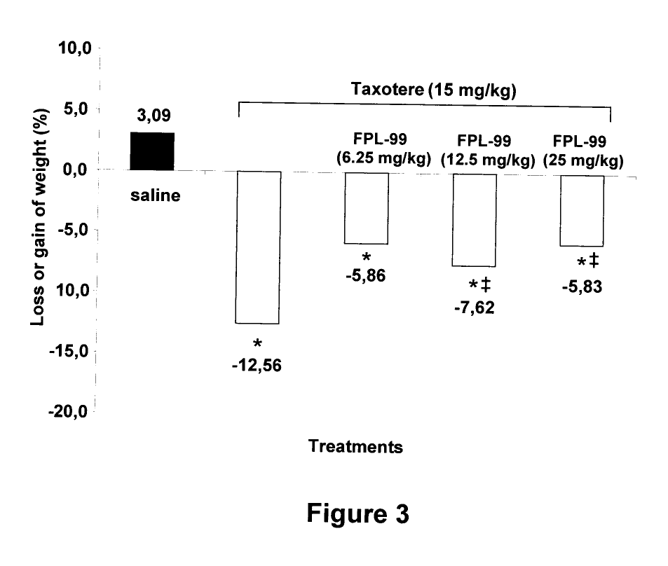

with 15 mg/kg of Taxotere®, decreased by about 13% the weight of mice. In contrast, the weight of mice treated with saline increased by about 3% (FIG. 2). Cachexia may be determined using the body weight of mice. In FIG. 3, β-caryophyllene (referred to as FPL-99) is shown to inhibit weight loss induced by Taxotere® by about 39 to 54% on day 7. Mice were considered cachectic if loss of weight was superior to 20% with regards to the initial weight. Table 2 below provides the number of mice having lost more than 20% of their initial weight on day 7. These results show that β-caryophyllene protects the mice against cachexia induced by Lewis lung tumours and Taxotere®.

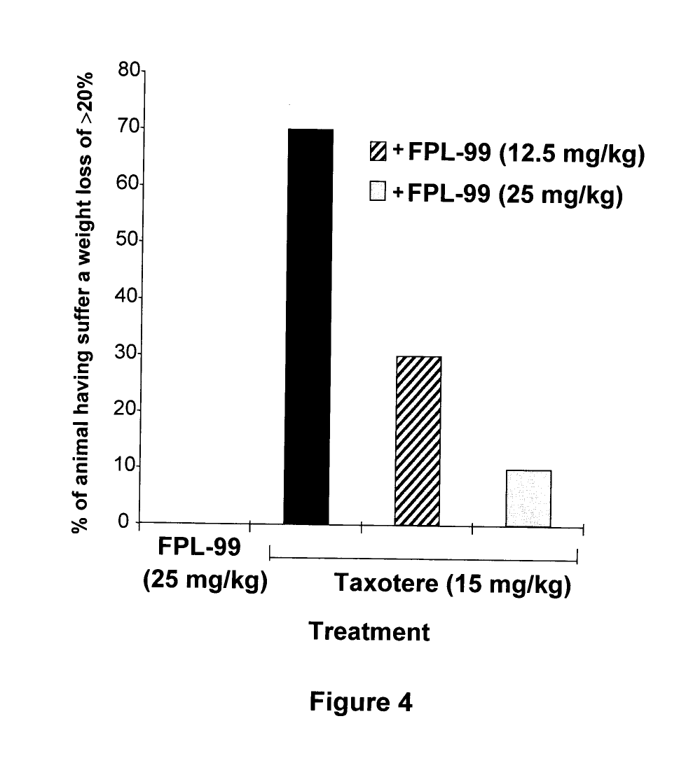

In a further experiment, the body weight of animals was measured every day during 18 days. FIG. 4 show the percentage of mouse having lost more than 20% of their weight during 18 days following treatment. β-caryophyllene (identified as FPL-99 on FIG. 4) treatment administered alone did not induce loss of weight. However, four days following treatment with 15 mg/kg of Taxotere®, 70% of mice had lost more than 20% of their initial weight. In contrast, β-caryophyllene (6.25 mg/kg, 12.5 mg/kg or 25 mg/kg) combined with 15 mg/kg of Taxotere® decreased significantly the loss of weight in comparison with Taxotere® only. Each experiment was performed on 10 mice.

### Example 2

### β-Caryophyllene Inhibits Cachexia Induced by Lewis Lung Tumor Alone

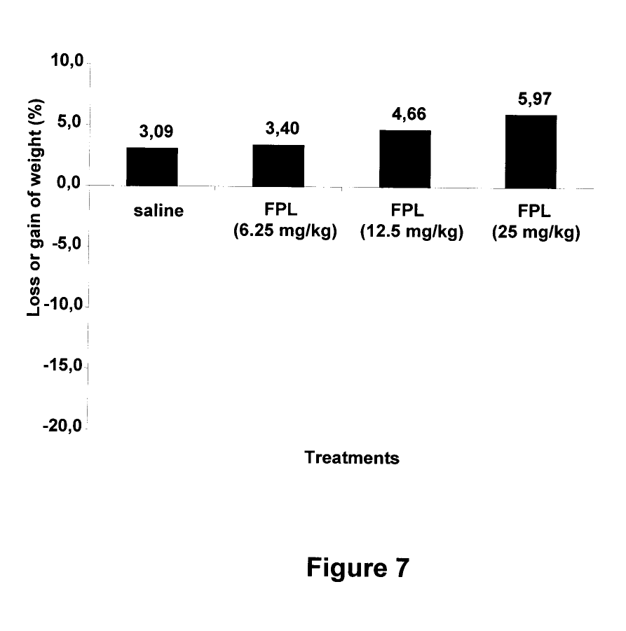

FIG. 7 presents the effect of β-caryophyllene alone on the body weight of tumor-bearing mice on day 7. It is important to note that the mice used in the above experiment were approximately eight weeks old (20-22 g) and were thus in growing phase. Moreover, the presence of tumor does not influence significantly the weight of mice on day 7. The results obtained show that β-caryophyllene (25 mg/kg, identified as FPL-99)) increased significantly weight gain by about 6% in comparison with 3% when the mice are treated with saline. Thus, β-caryophyllene induced weight gain in tumor bearing mice independently of the treatment with Taxotere®. Each experiment was performed on 10 mice.

### Example 3

### β-Caryophyllene Inhibits Anorexia Induced by Lewis Lung Tumor And Taxotere® in Tumor-Bearing Mice

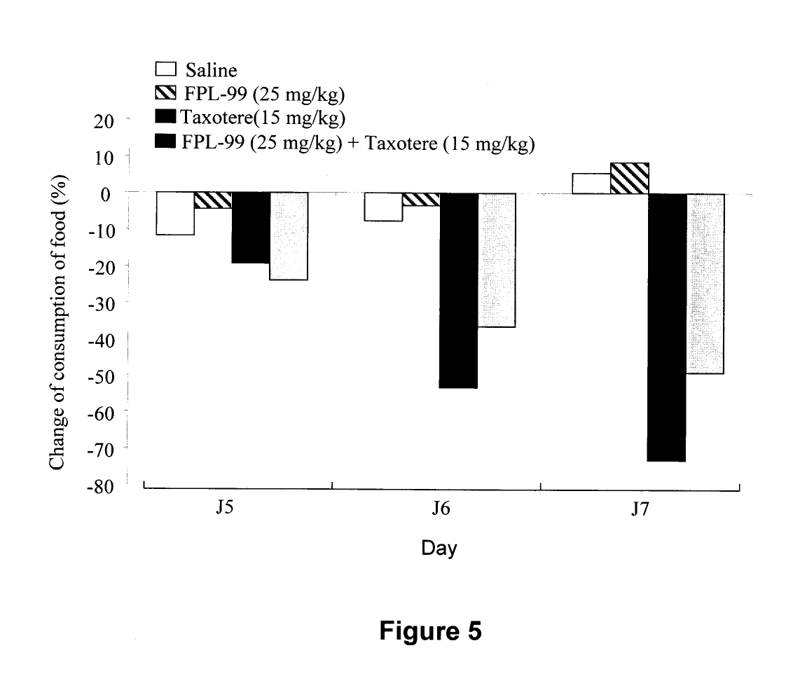

The effect of Taxotere® combined or not with β-caryophyllene on the consumption of food was assessed. The results presented in FIG. 5 are expressed as the percentage of change of consumption of food in comparison with day 1. The treatment with Taxotere® (15 mg/kg) caused anorexia decreasing the consumption of food by about 55% and 75% on day 6 and 7 respectively. β-caryophyllene (identified as FPL-99 on FIG. 5) treatment combined with Taxotere® improves the consumption of food. Indeed, β-caryophyllene reduced anorexia with an increase in food consumption of about 20% on day 6 and 25% on day 7, in comparison with Taxotere®-treated mice.

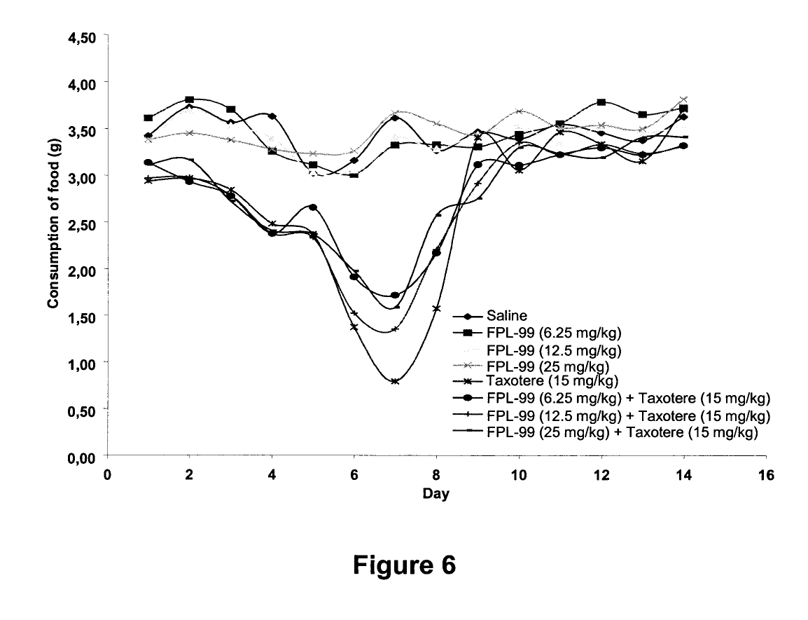

The effect of β-caryophyllene on consumption of food of mice was also evaluated during 18 days (FIG. 6).

### Example 4

### β-Caryophyllene Increases the Body Weight of Healthy Mice

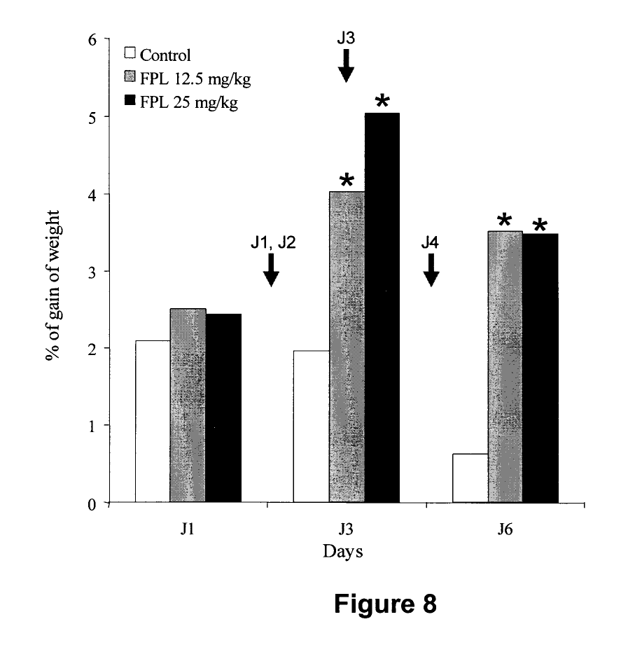

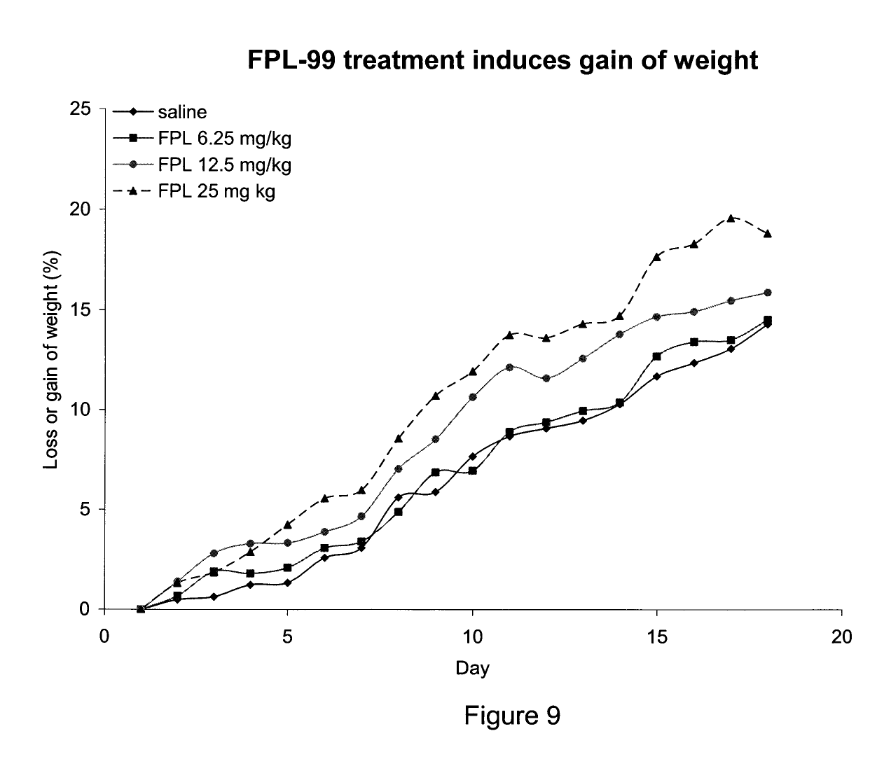

In another experiment, healthy mice (without tumor) were treated with vehicle or 12.5 or 25 mg/kg of β-caryophyllene on day 1 to 4. The body weight of mice was measured and the results were expressed as the percentage of loss or gain of weight with regards to initial weight. The results presented in FIGS. 8 and 9 are from 10 mice per treated group and show that β-caryophyllene (identified as FPL-99) increased significantly the percentage of weight gain in comparison with vehicle (without β-caryophyllene). On day 1, no significant difference was observed between treatment with vehicle and 12.5 or 25 mg/kg β-caryophyllene (with about 2% of weight gain). However, α-caryophyllene increased weight gain with regards to initial weight on day 3 by about 4% and 5% for 12.5 and 25 mg/kg, respectively, in comparison with 2% for the vehicle.

In conclusion, β-caryophyllene protected mice against anorexia induced by Lewis lung tumours and Taxotere®. Moreover, β-caryophyllene increased weight gain in Lewis lung tumours-bearing mice and in healthy mice.

### Example 5

### Effects of β-Caryophyllene on Cachexia Induced by Turpentine

The effect of β-caryophyllene on cachexia was evaluated using turpentine abscess mice model. Turpentine induces a local inflammation causing a drop of body weight. In the first experiment, the mice were treated orally with vehicle (olive oil and vitamin E (5 mg/ml)) or 50 mg/kg or 300 mg/kg of β-caryophyllene on day 1 to 3. On day 2, turpentine (Sigma-Aldrich, St-Louis) was administered subcutaneously (100 μl/mouse) in the hind of C57BL6 mice weighting 18 to 21 g (Charles Rivers, Canada). Ten mice per group were used for the above experiment.

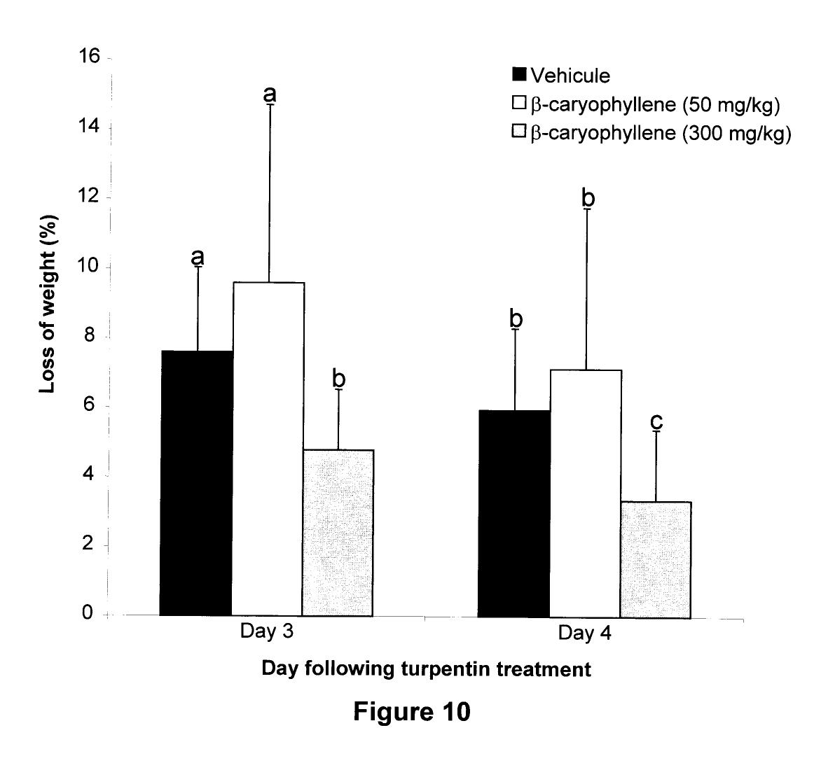

Mice weight was measured on day 1 to 4. Mice treated with vehicle (olive oil and vitamin E (5 mg/ml)) alone had a stable weight with regards to their initial weight on day 1 (data not shown). The results presented in FIG. 10 show that treatment with turpentine induced a significant loss of body weight of about 8% and 6% on day 3 and 4, respectively (Kruskal-Wallis One Way ANOVA on Ranks and post hoc Tukey test; p<0.001). Treatment with 50 mg/kg of β-caryophyllene did not affect significantly the loss of weight induced by turpentine on day 3 and 4 (RM One Way ANOVA and post hoc Student-Newman-Keuls Method; p>0.05). In contrast, treatment with 300 mg/kg of β-caryophyllene protected significantly the mice against loss of weight induced by turpentine on day 3 and 4 (p<0.05). Indeed, β-caryophyllene (300 mg/kg) decreased the loss of weight by about 37% on day 3 and 44% on day 4 in comparison with turpentine treated-mice.

### Example 6

### Effects of Monoterpenes and Sesquiterpenes on Cachexia Induced by Turpentine

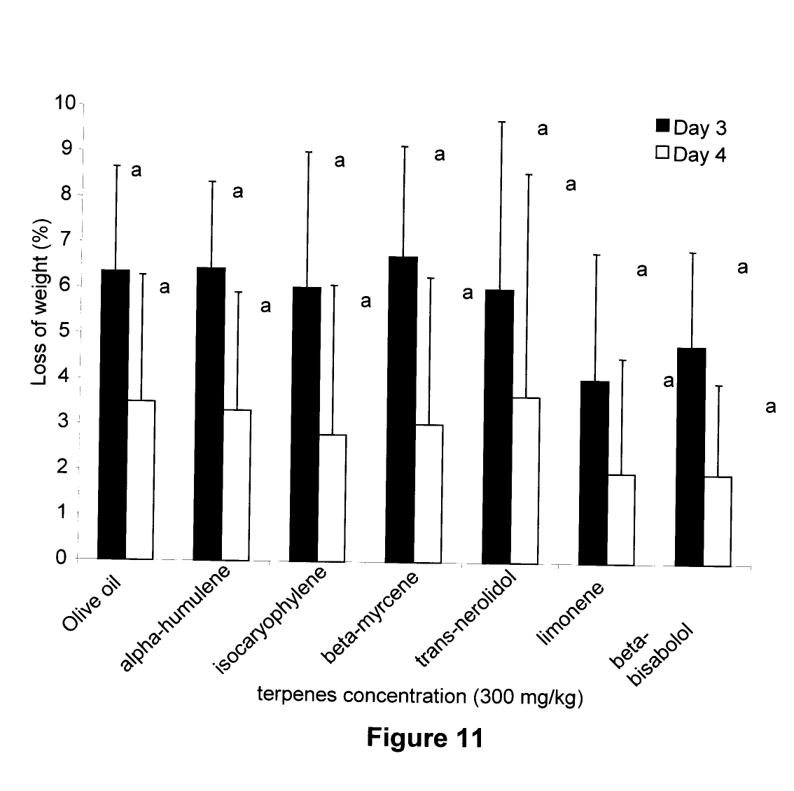

In a further experiment, the effect of monoterpenes (beta-myrcene, limonene) and sesquiterpenes (alpha-humulene, isocaryophyllene, trans-nerolidol, beta-bisabolol) was evaluated in the turpentine abscess model. The mice (n=10 for each group) were treated orally with vehicle (olive oil and vitamin E (5 mg/ml)) or with various terpenes (300 mg/kg) on day 1 to 3. On day 2, turpentine (Sigma-Aldrich, St-Louis) was administered subcutaneously (100 μl/mouse) in the hind of C57BL6 mouse weighting 18 to 21 g (Charles Rivers, Canada). Mice weight was measured on day 1 to 4. Mice treated with vehicle (olive oil and vitamin E (5 mg/ml)) without turpentine show a gain of weight of about 4% on day 3 and 5% on day 4 with regards to initial weight on day 1 (data not shown). The results presented in FIG. 11 show that treatment with turpentine induced a significant loss of body weight of about 6% and 3.5% on day 3 and 4, respectively (Kruskal-Wallis One Way ANOVA on Ranks and post hoc Tukey test; p<0.001). In FIG. 11, treatments with the tested monoterpenes and sesquiterpenes did not significantly protect the mice against the loss of weight induced by turpentine on day 3 and 4 (RM One Way ANOVA and post hoc Student-Newman-Keuls Method; p>0.05). Thus, among the monoterpenes and sesquiterpenes tested only β-caryophyllene shows a significant effect on body weight. Surprisingly, even alpha-humulene and isocaryophyllene, which are close structural variants of β-caryophyllene, do not show a protective effect with regards to weight loss in turpentine-induced cachexia. Thus, it appears that the effect of β-caryophyllene on body weight is very specific.

Although the present invention has been described hereinabove by way of specific embodiments thereof, it can be modified, without departing from the spirit and nature of the subject invention as defined in the appended claims.

### REFERENCES

1. Collado, I G et al., Recent advances in the chemistry of caryophyllene, Nat Prod Rep, The Merck Index, 11^{th} Ed., Rahway, N. J., Merck & Co, p. 287, 1989;
2. Daley, J. R., and Canada, T., Oncology Nutrition Connection, 12(4):
3. Guidance for Industry and Reviewers. Estimating the Safe Starting Dose in Clinical Trials for Therapeutics in Adult Healthy Volunteers, U.S. Department of Health and Human Services, Food and Drug Administration, Center for Drug Evaluation and Research (CDER), Center for Biologics Evaluation and Research (CBER), December 2002, Pharmacology and Toxicology;
4. Hendrickson, R. Ed. Remington: The Science and Practice of Pharmacy, 21st ed.; Lippincott Williams & Wilkins: Baltimore Md., 2005;
5. Inui A., Ca Cancer J. Clin., 52:72-97, 2002;
6. Muscaritoli M., et al., European J Cancer 42:31-41, 2006;
7. Nema, S. et al. Excipients and their use in injectable products, PDA J. of Pharm. Science and Technol., 51(4), 166-171 (1997);
8. Rowe et al., Handbook of pharmaceutical excipients, 2003, 4^{th} edition, Pharmaceutical Press, London UK;
9. Stewart, G. D., Skipworth, R. J E, Fearon, K. C H. (2006), Cancer cachexia and fatigue, Clinical Medicine, 6: 140-143;
10. Tisdale, M. J., Molecular pathways leading to cancer cachexia, Physiology (Bethesda), 20:340-8, October 2005.

1. (canceled)

2. A pharmaceutical composition for use in the prevention or treatment of Anorexia-Cachexia Syndrome, the pharmaceutical composition comprising purified β-caryophyllene together with a pharmaceutically acceptable carrier.

3-4. (canceled)

5. The composition of claim 2, wherein the Anorexia-Cachexia Syndrome is Cancer Anorexia-Cachexia Syndrome.

6-7. (canceled)

8. The composition of claim 2, wherein the Anorexia-Cachexia Syndrome comprises in addition to involuntary progressive weight loss, one or more of:
a) progressive loss of both fat and skeletal muscle,
b) refractoriness of weight loss to increased nutritional input,
c) elevated resting energy expenditure (REE),
d) decreased protein synthesis,
e) altered carbohydrate metabolism,
f) hyper-catabolism of muscle via the ATP-ubiquitin-proteasome pathway of proteolyis;
g) increased degradation of adipose tissue via lipolysis;
h) asthenia;
i) anemia,
j) chronic fatigue;
k) nausea; and
l) loss of bone mass.

9. The composition of claim 8, wherein the β-caryophyllene is substantially pure β-caryophyllene.

10-11. (canceled)

12. The composition of claim 9, wherein the composition further comprises one or more solubilizers selected from the group consisting of a PEG400, a derivative of castor oil and ethylene oxide, and polysorbate.

13. The composition of claim 12, wherein the composition further comprises one or more antioxidants selected from the group consisting of vitamin E, a hydrophilic vitamin E analog, alpha tocopherol acetate, butylated hydroxytoluene (BHT) and butylated hydroxyanisole (BHA).

14. The composition of claim 13, wherein the one or more antioxidants is 6-Hydroxy-2,5,7,8-tetramethylchroman-2-carboxylic acid.

15. The composition of claim 13, wherein the one or more antioxidants is vitamin E.

16-42. (canceled)

43. A method of 1) increasing weight gain; and/or 2) decreasing weight loss; and/or 3) increasing appetite in a subject in need thereof comprising administering to the subject an effective amount of a pharmaceutical composition comprising purified β-caryophyllene together with a suitable pharmaceutical carrier, whereby the subject experiences an increased weight gain; and/or a decreased weight loss; and/or an increased appetite.

44. A method of treating or preventing Anorexia-Cachexia Syndrome (ACS) comprising administering to a subject in need thereof a therapeutically effective amount of a composition comprising purified β-caryophyllene together with a suitable pharmaceutical carrier, whereby ACS is treated or prevented.

45-46. (canceled)

47. The method of claim 43, wherein the subject suffers from Cancer Anorexia-Cachexia Syndrome.

48-49. (canceled)

50. The method of claim 43, wherein the subject suffers from Anorexia-Cachexia Syndrome and wherein said Anorexia-Cachexia Syndrome comprises in addition to involuntary progressive weight loss, one or more of:
a) progressive loss of both fat and skeletal muscle,
b) refractoriness of weight loss to increased nutritional input,
c) elevated resting energy expenditure (REE),
d) decreased protein synthesis,
e) altered carbohydrate metabolism,
f) hyper-catabolism of muscle via the ATP-ubiquitin-proteasome pathway of proteolyis;
g) increased degradation of adipose tissue via lipolysis;
h) asthenia;
i) anemia,
j) chronic fatigue;
k) nausea; and
l) loss of bone mass.

51. The method of claim 50, wherein the β-caryophyllene is substantially pure β-caryophyllene.

52-53. (canceled)

54. The method of claim 50, wherein the composition is administered in combination with an antitumoral agent.

55-62. (canceled)

63. The method of claim 50, wherein the subject in need thereof is a subject which has lost more than 5% of his or her weight as compared to his or her weight prior to the onset of said Anorexia-Cachexia Syndrome.

64. The method of claim 50, wherein the subject in need thereof is a subject which has lost at least 10% of his or her weight as compared to his or her weight prior to the onset of said Anorexia-Cachexia Syndrome.

65-66. (canceled)

67. The method of claim 51, wherein the composition further comprises one or more solubilizers selected from the group consisting of a PEG400, a derivative of castor oil and ethylene oxide, and polysorbate and one or more antioxidants selected from the group consisting of vitamin E, a hydrophilic vitamin E analog, alpha tocopherol acetate, butylated hydroxytoluene (BHT) and butylated hydroxyanisole (BHA).

68. The method of claim 67, wherein the one or more antioxidants is 6-Hydroxy-2,5,7,8-tetramethylchroman-2-carboxylic acid.

69. The method of claim 67, wherein the one or more antioxidants is vitamin E.

70-71. (canceled)

72. The method of claim 67, wherein the one or more solubilizers is a derivative of castor oil and ethylene oxide.

73-81. (canceled)
